(19) United States
(12) Patent Application Publication
(10) Pub. No.: US 2005/0069594 A1
(43) Pub. Date: Mar. 31, 2005

(54) Pharmaceutical compositions and methods for the prevention of breast and ovarian cancer

(76) Inventors: Jan Lubinski, Szczecin (PL); Steven Narod, Toronto (CA); Elzbieta Kowalska, Szczecin (PL); Bohdan Gorski, Szczecin (PL); Tomasz Huzarski, Szczecin (PL); Tomasz Byrski, Szczecin (PL); Jacek Gronwald, Szczecin (PL); Jowita Huzarska, Szczecin (PL); Barbara Gawronska-Szklarz, Szczecin (PL)

Correspondence Address: John P. White, Esq.;Cooper & Dunham, LLP, 1185 Avenue of the Americas, New York, NY, 10036, US

(21) Appl. No.: 10/916,025

(22) Filed: Aug. 10, 2004

Related U.S. Application Data
(60) Provisional application No. 60/494,239, filed on Aug. 11, 2003.

(51) Int. Cl. A61K 35/78; A61K 33/04

(52) U.S. Cl. 424/702; 424/195.16; 424/755

(57) ABSTRACT

Disclosed is a method for the prevention of and the reduction of risk of breast cancer and ovarian cancer in a female human subject by the administration of selenium (or its salts or derivatives).

This application claims benefit of U.S. Provisional Application Ser. No. 60/494,239, filed Aug. 11, 2003, the contents of which are hereby incorporated by reference.

Various publications are referenced throughout this application. The content of these publications is incorporated herein by reference.

### BACKGROUND OF THE INVENTION

The invention provides a composition of selenium (or its salts or derivatives), methods for the prevention of breast/ovarian cancer in females with inherited high risk of cancer, particularly in carriers of BRCA1 gene mutations. BRCA1 (U.S. Pat. No. 5,654,155) and BRCA2 genes are related to high genetic predispositions to cancers. BRCA1 and BRCA2 genes have been cloned and, at present, their abnormalities can be detected at DNA and RNA levels. Carriers of mutations within above genes have high risk of breast and/or ovarian cancers. BRCA1 is the first gene recognized to be associated with high risk of breast and ovarian cancer (Miki et al, Science, 266-271, 1994). BRCA1 gene (GENBANK Accession Numbers: U14680 and 15595) contains 24 exons dispersed within 100 kbp of genomic DNA and its mRNA is of 7.8 kbp of the length. Intensive studies of this gene allowed to identify numerous BRCA1 mutations. For example, U.S. Pat. No. 5,693,473 is reporting large registry of such changes. WO 99/29903 is describing the next fifteen BRCA1 mutations. BRCA2 gene (GENBANK accession Number U43746) contains 27 exons dispersed within 70 kbp of genomic DNA and its mRNA is of 11-12 kbp of the length. Numerous mutations of BRCA2 gene have been reported as associated with cancer predisposition (for example WO 9928506).

All of described BRCA1 and BRCA2 gene mutations are available on page: http://www.nchgr.nih.gov/dir/lab-transfer/bic, the content of which is hereby incorporated by reference.

BRCA1 and BRCA2 mutations can be located in different exons. Increased frequency of selected particular mutations in families with breast/ovarian cancer aggregation has been recognized in some ethnic groups/populations such as Ashkenazi Jews among whom the high incidence of BRCA1 185delAG and 5382insC and BRCA2 6174delT are well known. In Poland, increased frequency of the following BRCA1 mutations has been reported: ex 2-185delAG, ex 5-300T→G, ex 5-309T→C, ex 11.15-3819delGTAAA, ex 11.17-4153delA, ex 20-5382insC (Polish patent P 335917). Opportunity of DNA testing based on analysis of limited number of mutations without significant loss of sensitivity in detection of abnormalities is creating a chance, valuable for several countries, of BRCA1/BRCA2 examination with cost-effectiveness extremely high. In Poland, around 100,000 of women are carriers of BRCA1 mutation.

Research spanning the last 25 years has established that selenium is effective in the reduction of cancer incidence when provided to animals at doses only 5- to 10-fold above nutritional requirement (El-Bayoumy K., The role of selenium in cancer prevention, Philadelphia, Lippincott, 1-15, 1991). Chemoprevention studies with selenium in animal model systems have indicated that this element is effective for most, if not all of the organ systems and is protective against the carcinogenic effects of a wide variety of insults (El-Bayoumy K., The role of selenium in cancer prevention, Philadelphia, Lippincott, 1-15, 1991). Both epidemiological studies and supplementation trials have also supported its efficacy in lowering the incidence of cancers of the liver, colon, prostate and lung (Yu S. Y. et al. Protective role of selenium against hepatitis B virus and primary liver cancer in Qidong, Biol Trace Elem Res, 56: 117-124, 1997, Clark L. C. et al. Effects of selenium supplementation for cancer prevention in patients with carcinoma of the skin. J Am Med Assoc, 276: 1957-1963, 1996, Yoshizawa K. et al. Study of prediagnostic selenium level in toenails and risk of advanced prostate cancer, J Natl Cancer Inst (Betsheda), 90: 1219-1224, 1998; Brooks J. D. et al. Plasma selenium level before diagnosis and risk of prostate cancer development, J Urol, 166: 2034-2038, 2001). The latest literature data are, however, not supporting negative correlation between the level of selenium in the body of patients and the risk of breast cancer.

Garland M. et al. concluded that diet supplementation with selenium does not influence the risk of female breast cancer (J. Am. Coll Nutr. 1993, Aug; 12(4): 400-11, J Natl Cancer Inst 1995, Apr 5; 87(7): 497-505). Similar conclusions have been reported by Ghadirian P. et al. (A case-control study of noenail selenium and cancer of the breast, colon and prostate, Cancer Detect Prev, 24: 305-13, 2000).

### DETAILED DESCRIPTION OF THE INVENTION

It has been recognized, recently, that breast cancer is a heterogenous disorder and around 10% of consecutive breast/ovarian cancers occurs as a result of inherited high genetic predisposition to cancers. Due to this, the aim of invention is to provide pharmaceutical compositions and methodology of their application allowing efficient lowering of the risk of breast/ovarian cancers in individuals with high genetic predisposition to these tumors, especially in carriers of BRCA1 gene mutations. Particular goal of invention is the provision of pharmaceuticals for chemoprevention of breast/ovarian cancers, which should be offered to females with constitutional mutations of BRCA1 gene.

Unexpectedly, the problem described as above has been solved, at least in large part, due to this invention. The subject of invention is pharmaceutical composition for lowering the risk of breast/ovarian cancer in person with inherited high risk of disease, especially in carrier of BRCA1 mutation, characterized by contents of selenium (its salt or derivatives) and, if necessary, pharmaceutically appropriate carrier.

It can be recommended that selenium is selected among its organic forms such as methylselenocysteine, methyloseleninic acid, selenomethinine, selenocysteine or inorganic forms such as selenium dioxide, selenonic acid, selenic acid and their salts. It can be recommended also that selenium originates from such natural products like Se-enriched yeast or broccoli. It can be recommended that selenium is a salt selected among: barium selenite, lithium selenite, calcium selenite and, what is particularly valuable, sodium selenite. Valuable pharmaceutical composition is an isotonic solution, containing selenium at concentration 0.1-10% w/w. It can be recommended to use as a dissolvent an aquaous solution of ethanol at concentration 10-96% w/w., distilled water, physiologic solution preferably buffered. It can be recommended as well if composition according to invention contains selenium salt in aquaous solution of ethanol.

The subject of invention is also the method of prevention of breast/ovarian cancer in person with inherited high genetic risk of tumor particularly in female carrier of BRCA1 characterized by supplementation of patient with compounds containing selenium (or its salts or derivatives), and using compositions as described above. It can be recommended that in methodology according to invention patient is supplemented with selenium at doses 50-1000 μg per day. It is valuable, in methodology according to invention, to use composition containing selenium after detection in patient the constitutional BRCA1 gene mutation.

Unexpectedly, increased sensitivity to bleomycin as measured using cytogenetic test according to Hsu was detected in BRCA1 mutation carriers (0.58 vs. 0.39 chromosome breaks per cell; p<10⁻⁴), that was normalized after 1-month supplementation of diet with selenium at doses around 300 μg per day.

Additionally, unexpectedly as well, the lowering of breast/ovarian cancer risk, was observed (see example 3):

For the period of 2 years, occurrence of new breast/ovarian cancers was analysed in the group of 30, initially healthy, carriers of BRCA1 mutation supplemented with ethanol solution of sodium selenite at dose 300 μg per day and in the control group of 30 BRCA1 carriers matched for mutation type and year of birth (average age—46.7 yrs). During above pilot studies, 1 breast cancer case was observed in the group supplemented with selenium and 4 breast cancers plus 1 ovarian cancer in the control group.

Additionally, for the period of 1 year, occurrence of new breast/ovarian cancers was analysed in the group of 200, initially unaffected, carriers of BRCA1 mutations supplemented with ethanol solution of sodium selenite at dose 300 μg per day and in the control group of 200 BRCA1 carriers matched for mutation type and age/average age 38.6 yrs). During above pilot studies 1 breast and 1 peritoneal cancers were observed in the group supplemented with selenium, whereas 2 breast and 2 ovarian cancers were diagnosed in the control group.

In order to present the sense of invention, the description of invention is expanded by examples 1-3. However, it is not our intention to introduce claims limited to situations described in examples, because basing on presented sense of invention combined with knowledge generally available, experts will be able to prepare other variants comprised in defined claims.

### EXAMPLE 1

### Compositions Containing Selenium

Basing on information available from pharmakopoeia, experts are able to prepare different variants of compositions containing selenium, especially selenium (IV). For example, selenium can be chosen from organic forms such as methylcysteine, methylseleninic acid, selenomethionine, selenocysteine or from inorganic forms such as selenium dioxide, selenic acid, (H₂SeO₃) or its salts. For example selenium (IV) salt valuable for preparation of compositions according to invention is sodium selenite. Natural products with high concentration of selenium such as selenium-enriched yeast or broccoli are attractive compounds as well.

Particularly valuable, although not the only one, composition according to invention are solutions of selenium at concentration 0.1-10% w/w, especially isotonic solutions. Such solutions can be made, for example, using such dissolvents as aquaous solution of ethanol at concentration 10-96% w/w, distilled water, physiologic solution especially if buffered.

It has been shown that the optimum of pharmacological effect can be achieved if daily dose is 50-1000 μg of selenium. The above values are modifiable depending on biological access of selenium applied to preparation of composition, as well depending on influence of associated compounds, pharmaceutical carriers.

Selected, although not the only ones, examples of such compositions are presented below:

A) Ethanol Solution of Sodium Selenite.

In order to prepare 1000 g of composition −2.03 g of sodium selenite (Na₂SeO₃) is dissolved in 334.32 g of H₂O. To aquaous solution of sodium selenite obtained as above 663.65 g of 96% aquaous solution of ethanol is added, and the solution is carefully mixed. Composition prepared in such way can be used for oral supplementation of patient, 2 times 10 drops per day, what corresponds to daily dose around 300 μg of pure selenium. It can be recommended to divide composition into bottles containing 11-12 g, because such amount covers one-month supplementation. Described composition is stable, maintains its form and activity at least 18 months if stored at 4° C. It can be used by patients without giving side effects.

B) Aquaous Solution.

Composition based on aqua can be prepared by replacement of ethanol by distilled water or physiologic solution (preferably buffered) in recipe as in A.

### EXAMPLE 2

### Increased Rates of Chromosome Breakage in BRCA1 Carriers are Reduced by Oral Selenium Supplementation

Study subjects were recruited from among the attendees of a single familial cancer clinic of the Hereditary Cancer Centre of the Pomeranian Academy of Medicine in Szczecin, Poland. Women were referred to this clinic because of a family history of breast or ovarian cancer. The women who participated in this study had previously been offered, and had consented to, genetic testing. Case women were recruited from among those who had been found to be carriers of a deleterious mutation in the BRCA1 gene. The most common mutation was the Polish founder mutation 5382 ins C (16 carriers) but there were 10 women with other BRCA1 mutations. Control subjects were recruited from among the family members of the carriers (cases), but who had been determined not to carry the deleterious mutation. It was possible to generate 26 case-control pairs. In 20 instances the control was the sister of the case and in six instances she was a more distant relative. Only healthy women were invited to participate in this study; women with a past history of breast, ovary or other form of cancer were excluded. Each woman provided a blood sample at some time during one year study.

Chromosome sensitivity to bleomycin was measured according to the method of Hsu et al. (Sensitivity to the genotoxic effects of bleomycin in humans; possible relationship to environmental carcinogenesis, Int J Cancer, 1989, 43, 403-409). Bleomycin (Nippon-Kayuka) was added to the cell culture five hours before the end of the culture at a concentration of 0.03 IU/ml. Conventional harvesting and Giemsa staining were made. For each subject, 100 consecutive euploid cells in metaphase were read (cells with overlapping chromosomes were excluded). Each chromatid aberration (excluding gaps) was scored as one breakpoint and each exchange-type aberration was scored as two breakpoints. The total number of breakpoints per 100 cells was recorded for each subject. Reading of coded slides was performed blindly by one of us. For twenty randomly selected cases the counts were determined independently by two cytogenetic technicians with good agreement. The mean number of breaks per cell was calculated after inspection of 100 cells. The mean level of chromosome breaks was compared in the 26 case-control pairs using the paired t-test.

The mean number of chromosome breaks per cell was 0.58 for the BRCA1 mutation carriers (range 0.34 to 0.73) and was 0.39 for the controls (range 0.28 to 0.62) (table 1). For 23 of the 26 pairs, the value for the carrier exceeded that for the related control, for two pairs the control value was the higher of the two and for one pair the two values were equal (p<0.0001; sign test). The mean difference between the two groups was highly significant (p<0.0001).

TABLE 1
Comparison of chromosomes breaks per cell in BRCA1 carriers and
matched controls.

| | |
|---|---|
| Case (carrier) | Control (non-carrier) |

| | | Chromosome | | Chromosome |
|---|---|---|---|---|
| Case ID | age | breaks per cell | age | breaks per cell |
| 1. | 22 | 0.58 | 22 | 0.41 |
| 2. | 43 | 0.61 | 54 | 0.55 |
| 3. | 29 | 0.34 | 33 | 0.44 |
| 4. | 39 | 0.51 | 36 | 0.62 |
| 5. | 21 | 0.48 | 31 | 0.31 |
| 6. | 24 | 0.57 | 20 | 0.49 |
| 7. | 45 | 0.60 | 51 | 0.40 |
| 8. | 47 | 0.52 | 55 | 0.52 |
| 9. | 51 | 0.61 | 29 | 0.44 |
| 10. | 24 | 0.66 | 22 | 0.42 |
| 11. | 48 | 0.57 | 28 | 0.35 |
| 12. | 24 | 0.65 | 29 | 0.28 |
| 13. | 23 | 0.65 | 22 | 0.42 |
| 14. | 31 | 0.73 | 29 | 0.46 |
| 15. | 31 | 0.52 | 34 | 0.32 |
| 16. | 23 | 0.52 | 38 | 0.36 |
| 17. | 44 | 0.59 | 35 | 0.36 |
| 18. | 59 | 0.48 | 58 | 0.30 |
| 19. | 53 | 0.54 | 61 | 0.29 |
| 20. | 58 | 0.59 | 50 | 0.34 |
| 21. | 44 | 0.51 | 46 | 0.38 |
| 22. | 22 | 0.67 | 27 | 0.45 |
| 23. | 34 | 0.60 | 30 | 0.28 |
| 24. | 34 | 0.71 | 36 | 0.41 |
| 25. | 18 | 0.59 | 21 | 0.28 |
| 26. | 27 | 0.64 | 31 | 0.35 |
| mean | 35.3 | 0.58 | 35.7 | 0.39 |

A second group of BRCA1 carriers was recruited from this clinic for the purpose of studying the effect of selenium supplementation on frequencies of chromosome breaks. Thirty-five women with BRCA1 mutations agreed to participate in this phase of the study. Blood samples were taken prior to the onset of selenium supplementation, and again at a time from one month to three months following the start of daily selenium supplementation. Prior to selenium supplementation, the mean number of induced chromosome breaks per cell was 0.63 (range 0.42 to 0.81). An oral selenium solution was provided to the study subjects which contained 690 μg of pure selenium, in the form of sodium selenite (Na₂SeO₃) per ml of 70% ethanol. Subjects were requested to consume 0.2 ml of the solution twice daily. A second blood sample was taken at a time from one month to three months after commencement of selenium supplementation and the bleomycin test was repeated. After a mean of 1.5 months of selenium supplementation, the mean number of chromosome breaks per cell was reduced to 0.40 (range 0.27 to 0.60). In every case, the post-supplementation level showed a decline from the baseline level (table 2) and the mean difference was highly significant (p<10⁻¹⁰).

TABLE 2
Comparison of chromosome breaks per cell in BRCA1 carriers before
and after selenium supplementation.

| | Before selenium | After selenium |
|---|---|---|
| Case ID | supplementation | supplementation |
| 12 | 0.65 | 0.60 |
| 15 | 0.52 | 0.40 |
| 18 | 0.48 | 0.36 |
| 24 | 0.71 | 0.49 |
| 25 | 0.59 | 0.30 |
| 26 | 0.64 | 0.39 |
| 27 | 0.63 | 0.29 |
| 28 | 0.66 | 0.44 |
| 29 | 0.66 | 0.32 |
| 30 | 0.42 | 0.33 |
| 31 | 0.55 | 0.32 |
| 32 | 0.65 | 0.38 |
| 33 | 0.67 | 0.37 |
| 34 | 0.65 | 0.30 |
| 35 | 0.55 | 0.32 |
| 36 | 0.60 | 0.32 |
| 37 | 0.74 | 0.43 |
| 38 | 0.55 | 0.43 |
| 39 | 0.61 | 0.27 |
| 40 | 0.57 | 0.36 |
| 41 | 0.59 | 0.27 |
| 42 | 0.65 | 0.44 |
| 43 | 0.62 | 0.34 |
| 44 | 0.67 | 0.52 |
| 45 | 0.67 | 0.50 |
| 46 | 0.63 | 0.58 |
| 47 | 0.77 | 0.42 |
| 48 | 0.57 | 0.32 |
| 49 | 0.62 | 0.31 |
| 50 | 0.58 | 0.46 |
| 51 | 0.70 | 0.52 |
| 52 | 0.59 | 0.34 |
| 53 | 0.71 | 0.47 |
| 54 | 0.81 | 0.51 |
| 55 | 0.65 | 0.45 |
| mean | 0.63 | 0.40 |

Summarising, oral selenium is a good candidate for chemoprevention in women who carry a mutation in the BRCA1 gene.

### EXAMPLE 3

### Supplementation of Female BRCA1 Mutation Carriers with Selenium is Reducing Significantly the Risk of Breast/Ovarian Cancers

Occurrence of breast/ovarian cancers in the group of 30, initially healthy, carriers of BRCA1 mutations taking orally ethanol solution of sodium selenite at dose 300 μg daily and in the control group of 30 BRCA1 carriers matched for mutation type and year of birth, was observed during two years. Average age in both groups was 46.5 years. During the period of observation 1 breast cancer case was diagnosed in the group supplemented with selenium and 4 breast cancers plus 1 ovarian cancer have been diagnosed in control group. (Tab. 3).

TABLE 3
Occurrence of breast/ovarian cancers in initially healthy carriers of
BRCA1 mutations after supplementation with selenium and in
matched controls.

| | | Studied group | | Control group | |
|---|---|---|---|---|---|
| | | Year of | Occurrence of | Year of | Occurrence of |
| Lp. | Mutation | birth | cancer | birth | cancer |
| 1 | 5382insC | 1929 | — | 1933 | — |
| 2 | 5382insC | 1947 | — | 1944 | — |
| 3 | 5382insC | 1947 | — | 1946 | breast cancer |
| 4 | 5382insC | 1947 | — | 1946 | — |
| 5 | 5382insC | 1950 | — | 1949 | — |
| 6 | 5382insC | 1951 | — | 1951 | — |
| 7 | C61G | 1952 | — | 1951 | — |
| 8 | C61G | 1955 | — | 1952 | — |
| 9 | 5382insC | 1954 | — | 1953 | — |
| 10 | 5382insC | 1954 | — | 1954 | breast cancer |
| 11 | 4153delA | 1956 | — | 1957 | ovarian cancer |
| 12 | 5382insC | 1956 | — | 1957 | — |
| 13 | 4153delA | 1966 | — | 1963 | — |
| 14 | 4153delA | 1959 | — | 1960 | breast cancer |
| 15 | 5382insC | 1942 | — | 1948 | breast cancer |
| 16 | 5382insC | 1960 | breast cancer | 1960 | — |
| 17 | C61G | 1962 | — | 1962 | — |
| 18 | 4153delA | 1963 | — | 1962 | — |
| 19 | C61G | 1963 | — | 1963 | — |
| 20 | C61G | 1963 | — | 1963 | — |
| 21 | C61G | 1964 | — | 1964 | — |
| 22 | 5382insC | 1965 | — | 1965 | — |
| 23 | 5382insC | 1966 | — | 1964 | — |
| 24 | 5382insC | 1966 | — | 1966 | — |
| 25 | 5382insC | 1967 | — | 1967 | — |
| 26 | 5382insC | 1956 | — | 1956 | — |
| 27 | 5382insC | 1943 | — | 1943 | — |
| 28 | 5382insC | 1963 | — | 1965 | — |
| 29 | C61G | 1968 | — | 1968 | — |
| 30 | 5382insC | 1955 | — | 1956 | — |

1. A method of preventing breast cancer or ovarian cancer in a female human subject with inherited risk of breast or ovarian cancer comprising administration to the female human subject of an effective amount of selenium or a pharmaceutically acceptable salt of selenium, so as to thereby prevent breast cancer or ovarian cancer.

2. The method of claim 1, wherein the female human subject is a carrier of a BRCA1 gene mutation.

3. The method of claim 1, wherein the effective amount is 50-1000 μg of selenium per day.

4. The method of claim 1, comprising administration of a pharmaceutical composition comprising selenium or a pharmaceutically acceptable salt of selenium and a pharmaceutically acceptable carrier.

5. The method of claim 1, wherein the selenium is selected from its organic or inorganic form.

6. The method of claim 5, wherein organic form of selenium is selenomethylcysteine, methylseleninic acid, selenomethionine or selenocysteine.

7. The method of claim 5, wherein the inorganic form of selenium is selenium dioxiale, selenic acid (H₂SeO₃).

8. The method of claim 1, wherein selenium is derived from natural products.

9. The method of claim 1, wherein natural product is a plant product.

10. The method of claim 9, wherein the plant product is broccoli or selenium-enriched yeast.

11. The method of claim 1, wherein the selenium is a selenium (IV) salt.

12. The method of claim 11, wherein the selenium (IV) salt is sodium-, barium-, lithium-, or calcium- of selenite.

13. The method of claim 12, wherein the selenium is sodium selenite.

14. The method of claim 4, wherein the pharmaceutical composition is an isotonic solution.

15. The method of claim 4, wherein the pharmaceutical composition contains selenium in solution at a concentration of 0.1-10% w/w.

16. The method of claim 4, wherein the pharmaceutical composition comprises an aqueous solution of ethanol at a concentration of 10-96% w/w in a pharmaceutically acceptable solvent.

17. The method of claim 16, wherein solvent is distilled water, physiologic solution, or buffered physiologic saline.

18. The method of claim 4, wherein the pharmaceutical composition contains aqueous an ethanol solution of selenium salt.

19. A method of lowering the risk of breast cancer or ovarian cancer in a female human subject with inherited risk of breast or ovarian cancer comprising administration to the female human subject of an effective amount of selenium or a pharmaceutically acceptable salt of selenium, so as to thereby lower the risk of breast cancer or ovarian cancer.

20. A method for determining whether a female human subject is in need of supplementation of selenium or a pharmaceutically acceptable salt of selenium comprising a step of determining whether the female human subject has a constitutional mutation in BRCA1 gene, wherein detection of the mutation indicates that the female human subject is in need of selenium or a pharmaceutically acceptable salt of selenium.
